(19) United States
(12) Patent Application Publication
(10) Pub. No.: US 2016/0361416 A1
(43) Pub. Date: Dec. 15, 2016

(54) WATER IN LIQUID CRYSTALLINE STATE AND IN RESONANCE WITH MATERIAL

(71) Applicant: Donna Lynn TAYLOR, Cambridge (CA)

(72) Inventor: Donna Lynn TAYLOR, Cambridge (CA)

(21) Appl. No.: 14/739,048

(22) Filed: Jun. 15, 2015

(51) Int. Cl. A61K 41/00; A61K 33/00

(52) U.S. Cl. CPC A61K 41/0004; A61K 33/00

(57) ABSTRACT

A composition includes an amount of potable water existing in a liquid crystalline state. The amount of potable water is resonant with a material. A measure of preservative is mixed with the amount of potable water existing in the liquid crystalline state.

### TECHNICAL FIELD

This document relates to the technical field of (and is not limited to) a composition including potable water existing in a liquid crystalline state and being resonant (in resonance with) with a material (and method for making same).

### BACKGROUND

A drug is, in the broadest of terms, a chemical substance (a composition) that has a biological effect on a patient (human or other animal). Foods may be classified as a drug because food has a physiological effect on a patient (any animal species). Drugs are used to enhance physical or mental well-being. Drugs may be used for a limited duration, or on a regular basis for chronic disorders.

### SUMMARY

It will be appreciated that there exists a need to mitigate (at least in part) at least one problem associated with the existing drugs (also called the existing technology). After much study of the known systems and methods with experimentation, an understanding of the problem and its solution has been identified and is articulated as follows:

Energy medicine (also called alternative therapies) involve the use of energy fields. There are two main types: biofield therapies and bioelectromagnetic-based therapies. Biofield therapies affect energy fields that purportedly surround and penetrate the human body. Some forms of energy therapy manipulate biofields by applying pressure and/or manipulating the body by placing the hands in, or through, these fields (examples include qigong, reiki, and therapeutic touch, etc.). Bioelectromagnetic-based therapies involve the use of electromagnetic fields, such as pulsed fields, magnetic fields, or alternating-current or direct-current fields.

More people are taking responsibility for their health treatments, and are turning to alternative health products and therapies. More people believe that natural products are generally less invasive with fewer side effects for the treatment of disease.

In general terms, what is needed is water fit for human consumption that promotes health (for a patient taking such water), in which the water has been processed (augmented) in such a way that the water is resonant with a material.

More specifically, what is needed is water fit for human consumption that promotes health, in which the water has been treated (augmented) with electromagnetic energy (such as, an electromagnetic frequency) associated with the material (such as, healthy biological material). In this manner, the water is resonant with the material (that is, the water has a vibrational resonance with the material).

The water has been augmented with the vibration of the material (such as, a healthy biological material), and in this manner, the body of the patient may use this processed water in such a way that the water may beneficially affect the state of health of the user.

To mitigate, at least in part, at least one problem associated with the existing technology, there is provided (in accordance with a major aspect) composition. The composition includes an amount of potable water existing in a liquid crystalline state. The amount of potable water is resonant with a material. A measure of preservative is mixed with the amount of potable water existing in the liquid crystalline state.

To mitigate, at least in part, at least one problem associated with the existing technology, there is provided (in accordance with a major aspect) a method. The method is for preparing a composition. The method of preparing the composition includes: (A) resonating an amount of potable water existing in a liquid crystalline state, with a material, and (B) mixing a measure of preservative with the amount of potable water existing in the liquid crystalline state.

Other aspects are identified in the claims.

Other aspects and features of the non-limiting embodiments may now become apparent to those skilled in the art upon review of the following detailed description of the non-limiting embodiments with the accompanying drawings.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 (SHEET 1 of 9 SHEETS) depicts a side view of an embodiment of a water-processing apparatus;

FIG. 2, FIG. 3A and FIG. 3B (SHEETS 2, 3 and 4 of 9 SHEETS) depict a front view of an embodiment of a frequency-producing apparatus and a side view of and embodiment of the water-processing apparatus of FIG. 1 for manufacturing, at least in part, a composition;

FIG. 4 (SHEET 5 of 9 SHEETS) depicts a side view of an embodiment of the water-processing apparatus of FIG. 1;

FIG. 5A and FIG. 5B (SHEET 6 of 9 SHEETS) depict side views of embodiments of a container;

FIG. 5C and FIG. 5D (SHEET 7 of 9 SHEETS) depict side views of embodiments of the container having the composition manufactured, at least in part, by the frequency-producing apparatus and the water-processing apparatus of any one of FIGS. 2, 3A and 3B;

FIG. 6 (SHEET 8 of 9 SHEETS) depicts a schematic view of an embodiment of a method for manufacturing the composition of FIG. 5C; and

FIG. 7 (SHEET 9 of 9 SHEETS) depicts a schematic view of an embodiment of a method for manufacturing the composition of FIG. 5C.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted.

Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, but well-understood, elements that are useful or necessary in commercially feasible embodiments are often not depicted to provide a less obstructed view of the embodiments of the present disclosure.

### LISTING OF REFERENCE NUMERALS USED IN THE DRAWINGS

100 composition

101 amount of potable water

102 amount of potable water existing in a liquid crystalline state

103 material

104 measure of preservative

105 pharmacological agent

106 volume of hexagonally structured water

200 container

202 lid

204 label

300 water-processing apparatus

400 frequency-producing apparatus

402 device probes

404 plate

500 method

502 to 522 operation

600 method

602 to 624 operation

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the invention is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described.

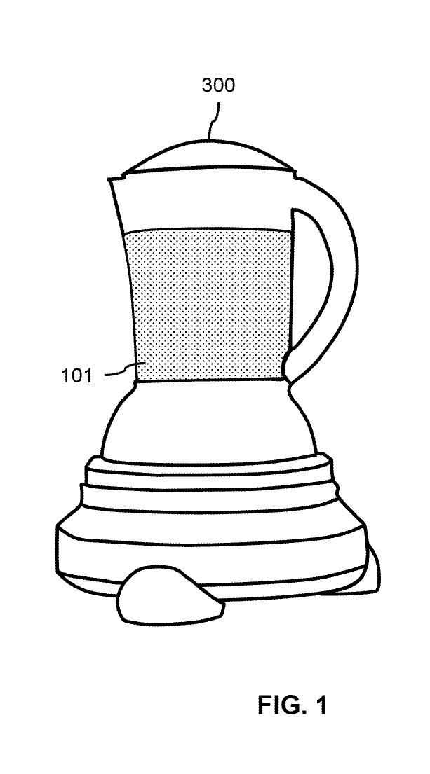

FIG. 1 depicts a side view of an embodiment of a water-processing apparatus 300.

Referring to the embodiment as depicted in FIG. 1, the water-processing apparatus 300 is configured to receive an amount 101 of potable water, and more preferably, to receive clean or purified potable water. The definition of potable water includes any type of potable fluid. In accordance with an option, before the amount 101 of potable water is received by the water-processing apparatus 300, the amount 101 of potable water is processed by a water-filtering system, a reverse-osmosis water purifier, and any equivalent thereof. The water filter is configured to remove impurities from the potable water by passing the potable water through a fine physical barrier, a chemical process and/or a biological process. The filter is configured to cleanse the potable water. Reverse osmosis is a water purification technology that uses a semipermeable membrane configured to remove larger particles from drinking water. In reverse osmosis, an applied pressure is used to overcome osmotic pressure, a colligative property that is driven by chemical potential, and a thermodynamic parameter. Reverse osmosis may remove many types of molecules and ions from solutions, including bacteria, and is used in the production of potable water.

The term “potable water” includes any type of potable material (potable substance) that may exist in any type of state (a fluid state, a solid state, a liquid state, and/or gas state, and any mixture thereof, and any equivalent thereof, etc.).

The term “potable” means any substance that is fit for (suitable for) consumption (ingestion) by a user (such as, a living matter, a human being, an animal, a bacteria, etc.).

Referring to the embodiment as depicted in FIG. 1, the water-processing apparatus 300 includes the VITALIZER PLUS (TRADEMARK) machine manufactured and/or provided by Vortex Water Tech., Inc. (located in California, USA). The VITALIZER PLUS (TRADEMARK) machine is configured to enhance drinking water quality by structuring the water into a hexagonal structure (that is, to produce hexagonally-structured water). Hexagonal water includes a cluster of water forming a hexagonal shape that is believed to enhance nutrient absorption, remove metabolic wastes, and enhance cellular communication, amongst other things.

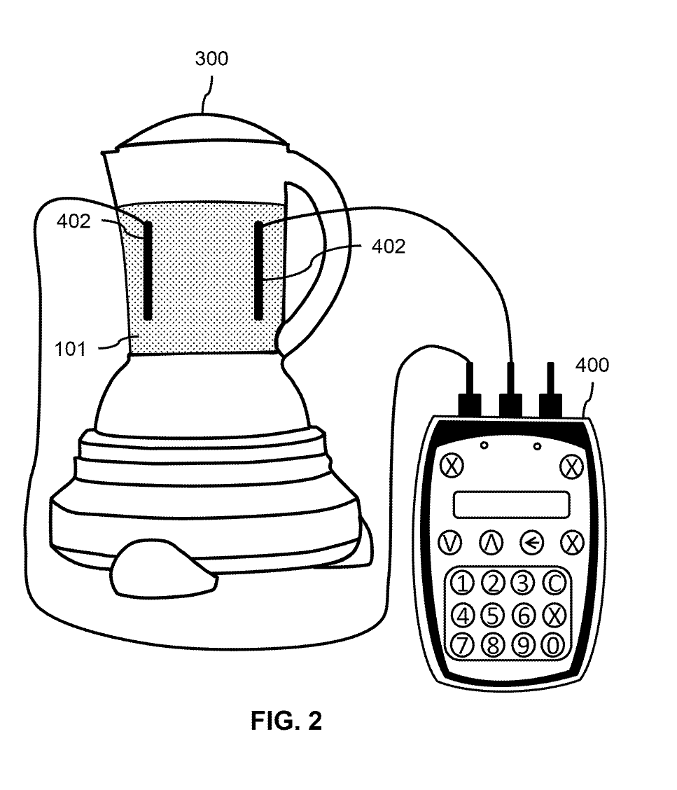

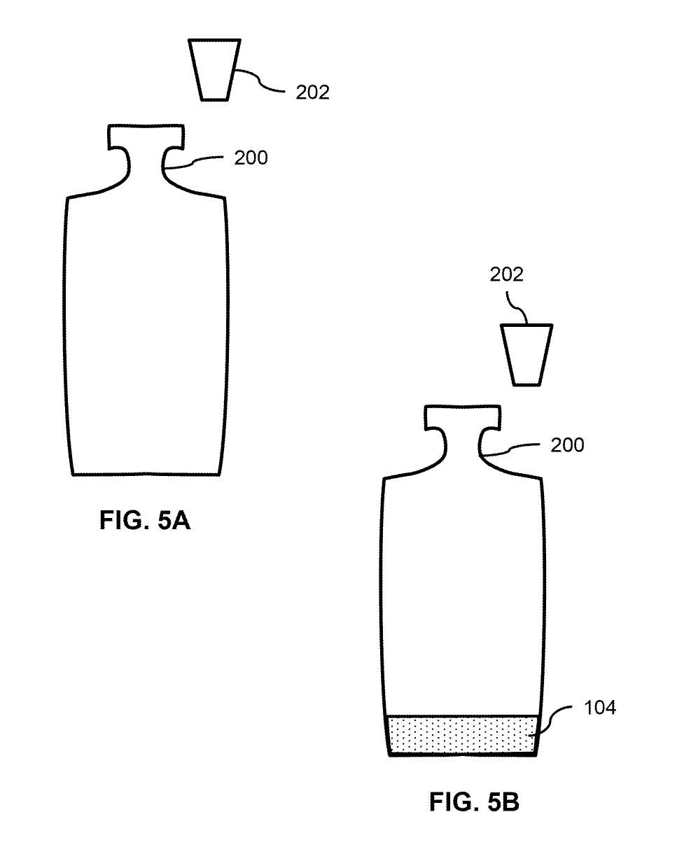

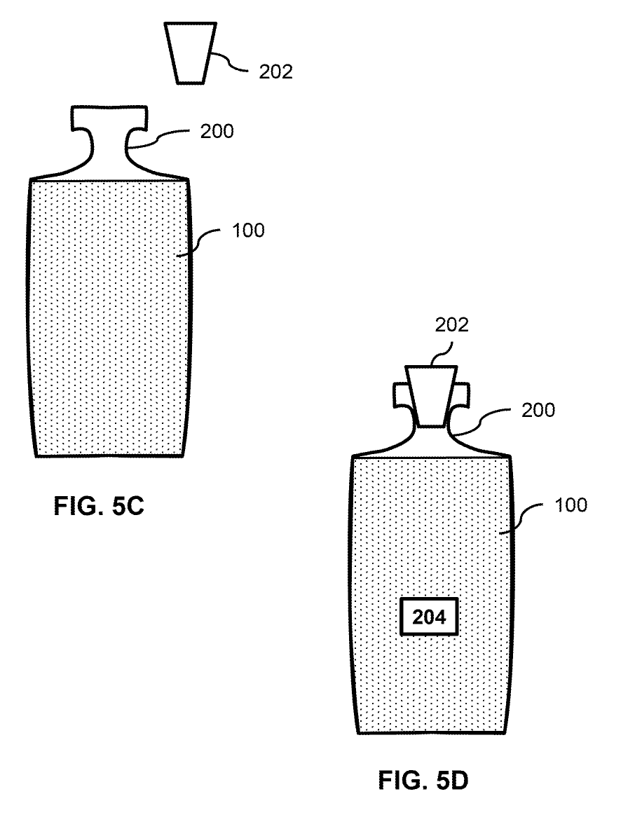

FIG. 2 depicts a front view of an embodiment of a frequency-producing apparatus 400 and a side view of an embodiment of the water-processing apparatus 300 of FIG. 1 for manufacturing, at least in part, a composition 100 (depicted in FIGS. 5C and 5D).

The composition 100 is configured to improve health (to improve health robustness) of a user once the user uses (ingests) the composition 100 (much like the user performing physical exercises for the purpose of enhancing or improving personal health). The composition 100 may be called a mixture. The composition 100 is not necessarily used (deployed) for treating a diseased condition or a pathological condition of a patient (that is, the composition 100 is not necessarily a medical treatment for treating the patient having a diseased condition or a pathological condition). The composition 100 beneficially affects (at least in part) the state of health of the user. The composition 100 assists in promoting the health of the user, similar to the health benefits that may be achieved by the user performing physical exercises or by the user eating clean, natural foods, etc.

Referring to the embodiment as depicted in FIG. 2, the water-processing apparatus 300 is configured to (in use or once so activated as depicted in either FIG. 3A or FIG. 3B) to convert the amount 101 of potable water to form the amount 102 of potable water (as depicted in FIG. 3A or FIG. 3B) that includes a volume 106 of hexagonally structured water existing in the liquid crystalline state.

The liquid crystalline state (of potable water or water) is called the fourth phase of water or the crystal phase, and may be found in a specific zone surrounding hydrophilic substances, such as human cells. The hydrophilic surfaces or interfaces may induce the water molecules to line up in a crystal formation. Zones of crystalline water may have unique measurable characteristics such as (and not limited to) molecular alignment, a negative electrical charge, greater viscosity, the ability to selectively exclude solutes and most importantly an enhanced ability to absorb various light frequencies. Water has four phases, and water in the liquid crystal phase stores energy. Water gets its energy from electromagnetic energy or light frequencies and, within this structured phase, like-charged entities may attract one another.

Liquid crystals are systems in which a liquid-like order exists in at least one direction of space and in which some degree of anisotropy is present. In other words, liquid crystals possess many of the mechanical properties of a liquid (such as high fluidity), but liquid crystals are not isotropic in their optical, mechanical, electric or magnetic properties. Liquid crystals do not refract light or conduct heat or electricity equally in all directions. Thus, liquid crystals have highly ordered electric and magnetic properties. Liquid crystals conduct light and electromagnetic information. One of the phases of liquid crystals, called nematic, may be compared with a ferromagnetic material, where the atoms, which are themselves relatively tiny magnets, are ordered so that they point in essentially the same direction giving them directional flow.

It will be appreciated that structured water may be produced by other devices. For instance, nature uses a combination of methods to structure water. When magnetic fields, turbulence creating vortical (relating to a vortex) movement and ionic minerals are present in and around cool water, the result is a stabilized molecular structure capable of holding informational patterns for a long period of time. Structured water is found naturally in various parts of the world, specifically in areas where the inhabitants are known to live exceptionally long lives. Nature uses movement to create vortices of energy. Natural curves along rocks and riverbed edges cause the water flowing there to spiral, forming a vortex within the water. The magnetic fields from the earth and the far-infrared energy from the sun along with the minerals from the rocks influence the water creating structured life enhancing water.

The term “hexagonal water” refers to an example of structured water. It is believed that drinking structured water may make a difference in health (health of a person or animal or living organism). It is believed that consuming hexagonal water increases the efficiency of metabolic processes, and that providing hexagonal water in the human body may increase vitality, slow the aging process and/or reduce the chances of disease.

It will be appreciated that any laboratory magnetic stirrer or magnetic mixer may be used to cause a vortical movement in water. A rotating magnetic field may be used to cause rotation of a stir bar immersed in the water placed in a beaker (creating a vortex in the water). A mineral may be added to the water in order to assist in the process of structuring the water. It is preferred to use the VITALIZER PLUS (TRADEMARK) device because this device is configured to produce hexagonally structured water in a way that is similar to principles used in nature (by utilizing magnetic and far infrared energy, adding oxygen and structure-making ionic minerals to a swirling vortex of potable water in order to produce energized hexagonally structured potable water). The water produced by the VITALIZER PLUS (TRADEMARK) device has been demonstrated to be hexagonally structured.

FIG. 3A and FIG. 3B depict a front view of an embodiment of the frequency-producing apparatus 400 (of FIG. 2) and a side view of an embodiment of the water-processing apparatus 300 of FIG. 1 for manufacturing, at least in part, the composition 100 (depicted in FIGS. 5C and 5D).

The frequency-producing apparatus 400 (of FIG. 3A or FIG. 3B) is configured to imprint structured potable water (hexagonally structured potable water) with an informational pattern, such as a frequency (one or more frequencies). More specifically, the frequency-producing apparatus 400 (of FIG. 3A or FIG. 3B) is configured to cause or urge the amount 102 of potable water to become resonant with a material 103 (such as, healthy biological material functioning with good health). Examples of the different types of material 103 are described below.

In accordance with a preferred embodiment, the frequency-producing apparatus 400 and the water-processing apparatus 300 are operated for a similar amount of time (duration of time). That is, the water-processing apparatus 300 is activated (to convert the amount 101 of potable water as depicted in FIG. 2) to form the amount 102 of potable water (as depicted in FIG. 3A or FIG. 3B) that includes the volume 106 of hexagonally structured water existing in the liquid crystalline state, while the frequency-producing apparatus 400 (of FIG. 3A or FIG. 3B) is configured to imprint structured potable water (hexagonally structured potable water) with the informational pattern.

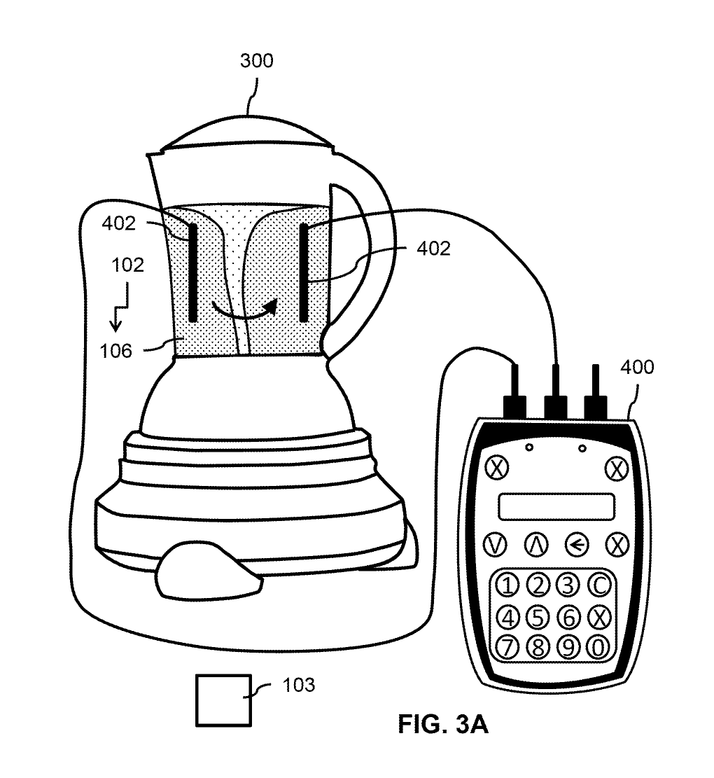

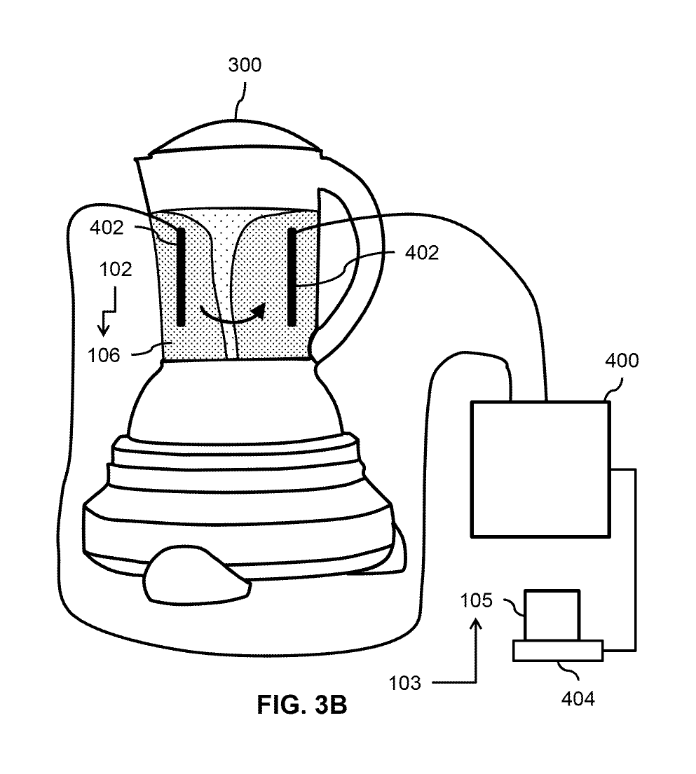

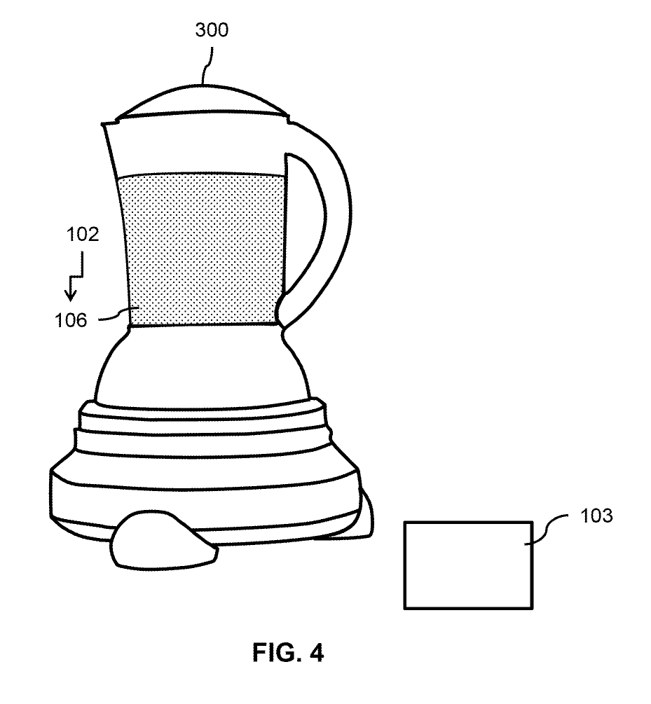

Once the frequency-producing apparatus 400 is deactivated, the device probes 402 of the frequency-producing apparatus 400 may be removed from the water-processing apparatus 300. The device probes 402 may be attached to the exterior or to the interior of the container of the water-processing apparatus 300. In accordance with the embodiment as FIG. 4, the water-processing apparatus 300 now contains the amount 102 of potable water existing in the liquid crystalline state, and the amount 102 of potable water is resonant with the material 103.

Water has a distinctive molecular structure that may be influenced by the electromagnetic environment that surrounds the water. Water may be formed into a repeating molecular pattern allowing the water to form a liquid crystal. Liquid crystals are a special state of matter because they have an orientation that is efficient at transferring signals. The geometrically repeating pattern (of structured water) provides a pathway for the flow of energy and information. However, unlike solid crystals, liquid crystals are more versatile since they are flexible and more responsive.

The structured pattern of the liquid crystal, and of water in a liquid crystal state, may be influenced by electric and/or magnetic fields. Since the human body is composed of about seventy percent water, it follows that human biology may also be affected by electric and magnetic fields. Alternating electrical current may be used to affect (change or impose or impart) the pattern orientation within a liquid crystal.

In a crystalline state, water aligns itself in a way that may conduct electricity. Most of the water in a healthy body is in the liquid crystalline state. In order to pass the information into the body from an external carrier source, there is either a continuum of the liquid crystal network or a transmitter and receiver relationship between the two structured sources. Many components of the body (such as the connective tissue, fascia, collagen and cell membranes) are known to be liquid crystals. The collagenous molecules, in which all of our organs and cells are encased, are arranged in highly regular arrays that conduct both electric current and information. Cells in the body are embedded in a liquid crystalline matrix. The body's connective tissue is an interconnected web through which electric information is conducted and processed. In this sense, the body is a polyphasic liquid crystal.

The human body is (functions as) an antenna system (that is, the human body receives and transmits electrical frequencies) because the crystalline matrix (of the human body) is capable of constantly transmitting and receiving energetic information. Electricity and/or light may be used to affect cells of the body. For instance, the transcutaneous electrical nerve stimulation (TENS) unit is configured to emit small amounts of electrical current to manage pain in the body. Electricity and/or magnetism are used extensively for medical diagnosis and/or treatment. Examples of this are the standard electrocardiogram (ECG) systems used to assess heart health (of a patient), and magnetic resonance imaging (MRI) systems used to assess the inside of the body (of a patient). Doctors use constructive interference wave mechanics to treat kidney stones in which doctors non-invasively focus a harmonic frequency on the kidney stone, which is a crystal, exploding the stone and allowing the stone to dissolve. The science of physics implies that the same harmonic resonance mechanism may enable similar electromagnetic frequencies to influence other cells and tissues in the body.

The term electromagnetic spectrum is used for the many different energy oscillations. The energies of the electromagnetic spectrum have different frequencies. The term frequency pertains to the number of cycles per second (CPS) or hertz (Hz) at which a wave vibrates or moves.

Living things and non-living things have a resonant frequency. A cell, a tissue and/or an organ within the body resonates at a particular resonant frequency in good health. These frequencies become altered when a cell, a tissue and/or an organ within the body exist in a disease state. For example, the lung vibrates at (has a resonant frequency of) 72 Hertz. If the lung develops an infection or becomes compromised, the resonant frequency of the lung may drop (become lower). Since the cells may be controlled by an electric current, it may be possible to introduce a healthy frequency (a resonant frequency associated with a state of health) to the lung in such a way that the lung may become rebalanced in response to the lung receiving the resonant frequency associated with a healthy lung. For the case where the resonant frequency of the healthy lung is provided to the diseased lung, the diseased lung may be urged to recuperate and attain normal healthy functioning. The body's tissues and organs emit electrical and electromagnetic signals that are unique to their type (resonant frequency). Applying electrical currents of similar frequencies may influence the function of the diseased tissues or cells. For example, some orthopedic surgeons apply electric currents to assist bone fracture union, and, in many cases, this method is able to successfully treat previously non-union cases (cases where there is a failure to heal the bone fracture completely). The body heals and rebalances itself when it is in resonance with its healthy frequencies (resonant frequency).

Specific electromagnetic frequencies may be used to affect the health of the human body in a beneficial way. Natural water (potable water) external to the body may be formed into a liquid crystal state that can be imprinted with electromagnetic frequencies. The pattern and information in the fluids and tissues of the body may be affected by liquid crystalline water programmed (imprinted) with specific electromagnetic frequencies (resonant frequencies).

Water may be used as an accumulator, a transmitter and/or a transducer of energy patterns. Water may imprint the electromagnetic information transmitted to it while the water is in an opened state forming into a liquid crystal, a state that may be induced by vortical movement. Water reacts to electromagnetic information in such a way that the water may retain the coherent energy pattern that is received (by the water) from the source of the electromagnetic information.

In accordance with a preferred embodiment, the frequency-producing apparatus 400 imparts, to the water, specific frequencies (at least one or more resonant frequencies) as the water is processed (such as, spun in a vortex) in the water-processing apparatus 300. The water-processing apparatus 300 is configured to further assist in the process of imprinting the resonant frequencies into the structured water. That structured water (once processed) holds the information (the resonant frequencies) intact in its structure (its memory). In this manner, the water may then transmit (once consumed by a patient) the information (the resonant frequencies) to another liquid crystalline system (to the patient or human tissues or cells). The technical effect of the preferred embodiment is that the specific signature frequencies (resonant frequencies) emitted by healthy human tissues may be impregnated into the structured water. In this manner, it may be possible to reprogram the state of health of diseased biological material (such as, human cells, etc.).

It will be appreciated that there are known devices configured to provide specific electromagnetic frequencies that correspond to the desired resonant frequencies. These devices may be used to impart the resonant frequencies to water as the water is formed into a liquid crystalline state. For instance, the Rife Digital electrotherapy device may be used to imprint the frequencies (resonant frequencies) that are supportive of healthy tissue (biological material) into the structured water by attaching the electrodes of the frequency-producing apparatus 400 to opposite exterior sides of the container (of the water-processing apparatus 300) holding the potable water. It will be appreciated that the electrodes of the frequency-producing apparatus 400 may be attached to the opposite interior sides of the container of the water-processing apparatus 300 (if so desired).

In accordance with an embodiment, the water that is made by the process may also be made by amplifying and transferring the frequencies of pharmacological agents (an example of an example of a frequency or information signal) into the structured water, in order to transmit the electromagnetic signature frequencies (the resonant frequencies) emitted by the pharmacological agents. In this manner, it is believed that the benefits of the pharmacological agents are provided by the specific frequency of the pharmacological agent without the harmful side effects typically associated with taking (ingesting) the molecular pharmacological agents.

It will be appreciated that the electromagnetic frequencies may pass through the plastic or glass medium of the container (of the water-processing apparatus 300) holding the potable water (an indirect transmission of the information), and that the electromagnetic frequencies (resonant frequencies) may be imprinted to the water. Of course, the transfer of electromagnetic fields and electric fields into the water may be performed indirectly or directly (such as, by transferring via electric current). Electromagnetic frequencies may travel through many objects (with some attenuation), such as plastics and glass.

It will be appreciated that the water imprinted with frequencies (the resonant frequencies) may transfer information to the patient once the patient ingests (receives) the water. It is believed that the patient may be beneficially affected by the composition 100 having the imprinted resonant frequencies (frequencies that resonate with healthy biological material). This process may be called resonance.

The liquid crystalline components of connective tissue within the body act as a coherent molecular antenna able to receive signals (such as, the resonant frequencies). The liquid crystalline structure of the water acts as a coherent molecular antenna able to transmit signals (the resonant frequencies).

For the case where two systems are oscillating at different frequencies, there is an impelling force called resonance that causes the two systems to transfer energy from one to the other. When two similarly tuned systems vibrate at different frequencies, there is another aspect of this energy transfer called entrainment that causes the systems to line up and to vibrate at the same frequency.

Biological systems function like radio sets, operating by co-resonance. A radio transmitter uses electromagnetic waves to transport information over a distance through a carrier medium. The radio receiver picks up those waves and converts the information into a usable form. The radio receiver uses electronic filters to separate out the specific desired frequency signal from all the other signals surrounding it. For instance, if a receiver is tuned to a radio transmitting frequency of about 108 MHz, the receiver and the transmitter vibrate at the same frequency. It is believed that biological cells function in this manner, that each cell is capable to tune into, pick up and vibrate to its own specific frequency (resonant frequency). In other words, each molecule, cell, tissue, and organ has an ideal resonant frequency that coordinates its activities (a frequency with which it is highly tuned and with which it is programmed to resonate).

FIG. 4 depicts a side view of an embodiment of the water-processing apparatus 300 of FIG. 1.

Once the frequency-producing apparatus 400 is deactivated, and the device probes 402 of the frequency-producing apparatus 400 are removed from the water-processing apparatus 300, the water-processing apparatus 300 contains the amount 102 of potable water existing in the liquid crystalline state. The amount 102 of potable water is resonant with the material 103.

FIG. 5A and FIG. 5B depict side views of embodiments of a container 200.

The container 200 (also called a vial) is configured to receiving (at least in part) the amount 102 of potable water (from the water-processing apparatus 300, as depicted in FIG. 4).

FIG. 5C and FIG. 5D depict side views of embodiments of the container 200 having the composition 100 manufactured, at least in part, by the frequency-producing apparatus 400 and the water-processing apparatus 300 of any one of FIG. 3A and FIG. 3B.

In accordance with an embodiment, the composition 100 (as depicted in FIG. 5C) is made by imprinting vibrational frequencies (resonant frequencies) into (onto) the vortically spun, hexagonally structured water. The composition 100 may act as an information carrier for a resonant frequency (resonant frequencies that are in resonance with the material 103, such as healthy biological material functioning with good health or vital health).

A technical effect of the process (for making the composition 100) is the preparation of potable water in such a way that the potable water carries information (a resonant frequency, at least one resonant frequency) into the patient. Water is used to carry resonant frequencies into the patient in such a manner that the biological material of the patient may respond and function in a healthy manner.

Referring to the embodiments as depicted in FIGS. 2 and 3A, the frequency-producing apparatus 400 includes the RIFE DIGITAL (TRADEMARK) machine supplied by the Rife Machine Store (located in Florida, USA), and manufactured and licensed by the Rife Machine Deutschland (located in Berlin, Germany). The RIFE DIGITAL (TRADEMARK) machine is configured to produce an adjustable frequency of about 0.01 KHz (kilo Hertz) to about 999.99 KHz at 16 volts DC (Direct Current). Dr. Royal Rife was the designer of the RIFE machine (which he called the RIFE frequency device). The frequency-producing apparatus 400 is configured to be connected to a set of device probes 402 (electrodes).

In general terms, the frequency-producing apparatus 400 is configured to impart (transmit) an electromagnetic frequency (also called a resonance frequency and/or a vibration having the resonance frequency) to the water held (being processed) by the water-processing apparatus 300. For instance, the vibration may be imparted to the water by a tuning fork (if so desired). The electromagnetic frequency may include a predetermined electromagnetic frequency or a set of predetermined electromagnetic frequencies. The electromagnetic frequency may have an amplitude of a predetermined value (magnitude).

The amount 102 of potable water (that is held in the water-processing apparatus 300) exists in the liquid crystalline state. The amount 102 of potable water is exposed to (receives) an electromagnetic frequency (emitted or transmitted by the frequency-producing apparatus 400). The electromagnetic frequency is resonant with (in resonance with) the material 103. The material 103 is associated with (has) a resonant frequency.

Resonance is a phenomenon in which a given system (such as, potable water, etc.) is driven by a vibrating system (an external force, such as a resonating device). This is done in such a way that the given system oscillates (with relatively greater amplitude) at a preferential frequency (or frequencies). In this manner, the potable water may store vibrational energy (also called, information). The resonance phenomena may occur with any type of vibration or wave energy.

In accordance with the embodiment as depicted in FIG. 3A and FIG. 3B, the volume 106 of hexagonally structured water is exposed to the electromagnetic frequency (as emitted or transmitted by the frequency-producing apparatus 400) during formation of the volume 106 of hexagonally structured water.

By way of example, the material 103 includes a biological material (also called, healthy biological material). The biological material includes (for instance) a biological tissue (also called, a healthy biological tissue). The healthy biological tissue is a cellular organizational level intermediate between cells and a complete organ. A tissue is an ensemble of similar cells from the same origin that together carry out a specific function. Organs are formed by the functional grouping together of multiple tissues. By way of another example, the biological material includes a biological cell (also called, a healthy biological cell). In accordance with an example, the biological material includes a biological organ (also called, a healthy biological organ). In accordance with an example, the biological material includes a biological system (also called, a healthy biological system). An example of the healthy biological system includes a body (of a healthy person or animal species).

Referring to the embodiment as depicted in FIG. 3B, the frequency-producing apparatus 400 includes the MORA III 200 MED TRONIK (TRADEMARK) amplifier manufactured and/or supplied by Med-Tronik GmbH (located in Friesenheim, Germany). A plate 404 (such as, a brass plate) is in contact with the material (such as, a drug material or a pharmacological drug). The plate 404 is attached to an input of the MORA III 200 MED TRONIK (TRADEMARK) amplifier. The output of the Mora III 200 Med Tronik amplifier is attached to the device probes 402 (brass electrodes). The device probes 402 are connected to (make contact with, either directly or indirectly) the potable water held by the water-processing apparatus 300, as depicted in FIG. 3B.

In accordance with an embodiment, the amount 102 of potable water (held in the water-processing apparatus 300) exists in the liquid crystalline state. The amount 102 of potable water is resonant with a frequency associated with a pharmacological agent 105 (which is an example of the material 103). The material 103 is a substance capable of emitting a specific frequency to be amplified and transmitted, such as the pharmacological agent 105 (such as a drug), a biological material, etc.

Referring to the embodiment as depicted in FIG. 5B, the measure 104 of preservative includes alcohol. The measure 104 of preservative is configured to sterilize and preserve the amount 102 of potable water existing in the liquid crystalline state.

Referring to the embodiment as depicted in FIG. 5C, the composition 100 includes (and is not limited to) the amount 102 of potable water existing in a liquid crystalline state. The amount 102 of potable water is resonant (in resonance) with a material 103 (as depicted in FIG. 3A or FIG. 3B) as a result of using the method 500 or the method 600.

As depicted in FIG. 5C, the composition 100 includes (and is not limited to) an amount 102 of potable water existing in a liquid crystalline state (as depicted in FIG. 4). The amount 102 of potable water is resonant (in resonance) with a material 103 (as depicted in FIG. 4). The composition 100 also includes the measure 104 of preservative (as depicted in FIG. 5B) mixed with the amount 102 of potable water existing in the liquid crystalline state.

In accordance with an embodiment, the composition 100 consists of: (A) the amount 102 of potable water (existing in the liquid crystalline state), with the amount 102 of potable water being resonant with the material 103, and (B) the measure 104 of preservative being mixed with the amount 102 of potable water (existing in the liquid crystalline state).

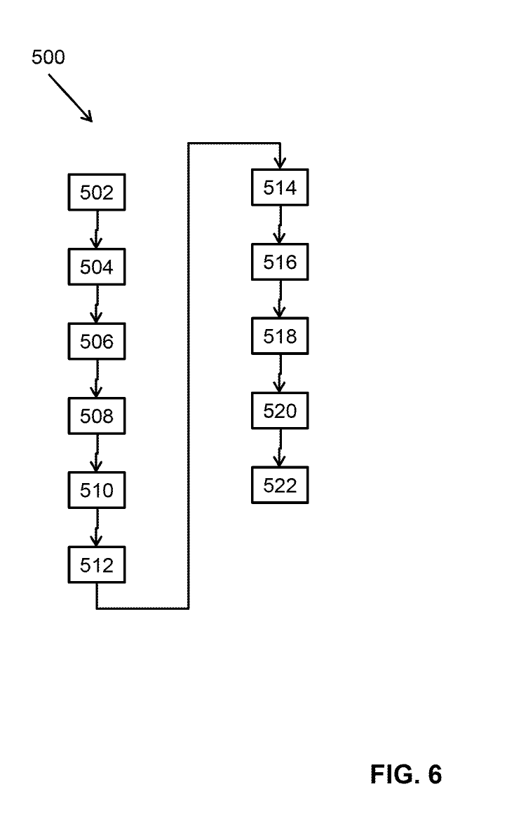

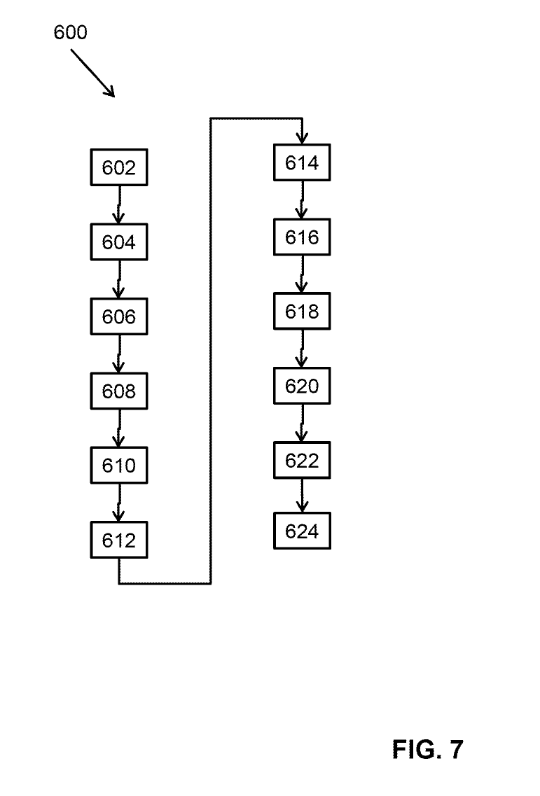

It will be appreciated that, in view of the foregoing, a method is provided for preparing the composition 100. Specific embodiments of the method are depicted in FIG. 6 and FIG. 7. In general terms, the method includes (and is not limited to): (A) resonating the amount 102 of potable water (existing in a liquid crystalline state) with a material, and (B) mixing the measure 104 of preservative (as depicted in FIG. 5B) with the amount 102 of potable water existing in the liquid crystalline state (as depicted in FIG. 4).

The composition 100 is prepared by a process (such as, the process associated with the method 500 and the method 600). The process includes the steps of: (A) forming the amount 102 of potable water in such a way that the amount 102 of potable water exists in a liquid crystalline state; (B) resonating the amount 102 of potable water existing in the liquid crystalline state with a material 103; and (C) mixing a measure 104 of preservative with the amount 102 of potable water existing in the liquid crystalline state.

FIG. 6 depicts a schematic view of an embodiment of a method 500 for manufacturing the composition 100 of FIG. 5C.

Referring to the embodiment as depicted in FIG. 6, the method 500 includes operation 502 to operation 522.

Operation 502 includes placing clean water (preferably, water treated by a reverse osmosis process, etc.) into the water-processing apparatus 300, as depicted in FIG. 1.

Operation 504 includes coupling the frequency-producing apparatus 400 (such as the device probes 402) to the water held by the water-processing apparatus 300, as depicted in FIG. 3A.

Operation 506 includes programming the frequency-producing apparatus 400 with a resonant frequency for the particular type of the composition 100 to be manufactured, as depicted in FIG. 3A. For example, for the case where the composition 100 is to be used for the process of beneficially affecting the liver (weather or not the liver is compromised, diseased or healthy), the resonant frequency includes a set of resonant electromagnetic frequencies including about 15.2 Hz, about 63.5 Hz, about 91.0 Hz, about 337.0 Hz, about 528.0 Hz, about 751.0 Hz, and about 787.0 Hz. Preferably, the frequency-producing apparatus 400 pulses (in use) the resonant electromagnetic frequencies simultaneously into the water held by the water-processing apparatus 300. The resonant electromagnetic frequencies vary depending on the target biological tissue (such as, an organ).

Operation 506 includes activating the frequency-producing apparatus 400 to emit the resonant frequency (as depicted in FIG. 3A) in such a way that the water held by the water-processing apparatus 300 may receive the resonant frequency.

Operation 508 includes activating the water-processing apparatus 300 in such a way that the water held by the water-processing apparatus 300 may be structured (as depicted in FIG. 3A). For instance, the water held by the water-processing apparatus 300 may be spun for about 18 minutes.

Operation 510 includes deactivating the water-processing apparatus 300 and the frequency-producing apparatus 400, as depicted in FIG. 3A.

Operation 512 includes disconnecting the electrodes of the frequency-producing apparatus 400, as depicted in FIG. 4.

Operation 514 includes cleaning and preparing a container 200, such as a 30 ml (milliliters) capacity fluid vial with a lid 202, as depicted in FIG. 5A.

Operation 516 includes adding (dispensing) the measure 104 of preservative (such as, about 9 ml of 27% alcohol) into the container 200 (as depicted in FIG. 5B). For example, a gluten free potato vodka or brandy may be used (if so desired). The addition of alcohol at the concentration designated may act as a sterilizing agent within the composition 100.

Operation 518 includes dispensing (as depicted in FIG. 5C) the processed water from the water-processing apparatus 300 of FIG. 4 into the container 200 of FIG. 5B. For instance, about 18 ml of the processed water may be dispensed into the container 200 of FIG. 5B.

Operation 520 includes installing the lid 202 (stopper) into the container 200 (as depicted in FIG. 5D). Preferably, a security seal or a non-tampering seal (known and not depicted) is configured to securely connect the lid 202 to the container 200.

Operation 522 includes applying a label 204 (as depicted in FIG. 5D) to the container 200.

In accordance an embodiment, the method 500 is used to manufacture the composition 100 received in the container 200, in which the composition 100 includes vortically spun reverse osmosis water (potable water) imprinted with various electromagnetic resonant frequencies of (associated with) a healthy biological material (an example of the material 103 is depicted in FIG. 3B) in combination with the measure 104 of preservative.

FIG. 7 depicts a schematic view of an embodiment of a method 600 for manufacturing the composition 100 of FIG. 5C.

Referring to the embodiment as depicted in FIG. 7, the method 600 includes operation 602 to operation 624. The method 600 is for manufacturing the composition 100 (by using pharmacological resonant frequencies).

Operation 602 includes placing clean water (preferably, water treated by a reverse osmosis process, etc.) into the water-processing apparatus 300, as depicted in FIG. 1.

Operation 604 includes coupling the frequency-producing apparatus 400 (such as the device probes 402) to the water held by the water-processing apparatus 300, as depicted in FIG. 3B. More specifically, for operation 604, the frequency-producing apparatus 400 includes the plate 404 (such as, a brass plate) that is in contact with the drug material. The plate 404 is attached to an input of the MORA III 200 MED TRONIK (TRADEMARK) amplifier. The MORA III 200 MED TRONIK (TRADEMARK) amplifier is manufactured and/or supplied by Med-Tronik GmbH (located in Friesenheim, Germany). The output of the MORA III 200 MED TRONIK (TRADEMARK) amplifier is attached to the device probes 402 that are connected to (make contact with, either directly or indirectly) the water held by the water-processing apparatus 300, as depicted in FIG. 3B.

Operation 606 includes activating the water-processing apparatus 300 (to spin the water). The water-processing apparatus 300 is operated in such a way that the water held by the water-processing apparatus 300 may be structured (as depicted in FIG. 3B).

Operation 608 includes activating the frequency-producing apparatus 400 (the amplifier). The amplifier is set to have a total gain of about 1 to about 480, using periods of about 7 seconds of amplification with pauses from about 3 seconds for a total of about 15 minutes. The frequency-producing apparatus 400 emits the resonant frequency (as depicted in FIG. 3B) in such a way that the water held by the water-processing apparatus 300 may receive the resonant frequency.

Operation 612 includes deactivating the frequency-producing apparatus 400 and the water-processing apparatus 300.

Operation 614 includes disconnecting the device probes 402 of the frequency-producing apparatus 400 (as depicted in FIG. 4).

Operation 616 includes cleaning and preparing the container 200, such as a 30 ml (milliliters) capacity fluid vial with a lid 202, as depicted in FIG. 5A.

Operation 618 includes adding (dispensing) the measure 104 of preservative (such as, about 9 ml of 27% alcohol) into the container 200 (as depicted in FIG. 5B). For example, a gluten free potato vodka or brandy may be used (if so desired). The addition of alcohol at the concentration designated may act as a sterilizing agent within the composition 100.

Operation 620 includes dispensing (as depicted in FIG. 5C) the processed water from the water-processing apparatus 300 of FIG. 4 into the container 200 of FIG. 5B. For instance, about 18 ml of the processed water may be dispensed into the container 200 of FIG. 5B

Operation 622 includes installing the lid 202 (stopper) into the container 200 (as depicted in FIG. 5D). Preferably, a security seal is configured to securely connect the lid 202 to the container 200.

Operation 624 includes applying a label 204 (as depicted in FIG. 5D) to the container 200.

In accordance with the method 600, the composition 100 (received by the container 200) includes vortically spun reverse osmosis water (potable water) imprinted with various pharmacological electromagnetic frequencies (associated with a pharmacological material or a drug composition, which is an example of the material 103 depicted in FIG. 3B) in combination with the measure 104 of preservative.

This written description uses examples to disclose the invention, including the best mode, and also to enable any person skilled in the art to make and use the invention. The patentable scope of the invention is defined by the claims, and may include other examples that occur to those skilled in the art. Such other examples are within the scope of the claims if they have structural elements that do not differ from the literal language of the claims, or if they include equivalent structural elements with insubstantial differences from the literal language of the claims.

It may be appreciated that the assemblies and modules described above may be connected with each other as required to perform desired functions and tasks within the scope of persons of skill in the art to make such combinations and permutations without having to describe each and every one in explicit terms. There is no particular assembly or component that may be superior to any of the equivalents available to the person skilled in the art. There is no particular mode of practicing the disclosed subject matter that is superior to others, so long as the functions may be performed. It is believed that all the crucial aspects of the disclosed subject matter have been provided in this document. It is understood that the scope of the present invention is limited to the scope provided by the independent claim(s), and it is also understood that the scope of the present invention is not limited to: (i) the dependent claims, (ii) the detailed description of the non-limiting embodiments, (iii) the summary, (iv) the abstract, and/or (v) the description provided outside of this document (that is, outside of the instant application as filed, as prosecuted, and/or as granted). It is understood, for this document, that the phrase “includes” is equivalent to the word “comprising.” The foregoing has outlined the non-limiting embodiments (examples). The description is made for particular non-limiting embodiments (examples). It is understood that the non-limiting embodiments are merely illustrative as examples.

1. A composition, comprising:
an amount of potable water existing in a liquid crystalline state, and the amount of potable water being resonant with a material; and
a measure of preservative being mixed with the amount of potable water existing in the liquid crystalline state; and
wherein an electromagnetic frequency, produced by an electromagnetic information source, was imparted to the amount of potable water while the amount of otable water was bein converted into the liquid crystalline state in such a way that the structured water was imprinted with and retains the coherent energy pattern that was received from the electromagnetic information.

2. The composition of claim 1, wherein:
the amount of potable water existing in the liquid crystalline state is exposed to an electromagnetic frequency; and
the electromagnetic frequency is resonant with the material.

3. The composition of claim 2, wherein:
the amount of potable water includes a volume of hexagonally structured water existing in the liquid crystalline state.

4. The composition of claim 3, wherein:
the volume of hexagonally structured water is exposed to the electromagnetic frequency during formation of the volume of hexagonally structured water.

5. The composition of claim 2, wherein:
the electromagnetic frequency includes a predetermined electromagnetic frequency.

6. The composition of claim 2, wherein:
the electromagnetic frequency includes a set of predetermined electromagnetic frequencies.

7. The composition of claim 2, wherein:
the electromagnetic frequency has an amplitude of a predetermined value.

8. The composition of claim 1, wherein:
the amount of potable water exists in the liquid crystalline state, and the amount of potable water is resonant with a frequency associated with a pharmacological agent.

9. The composition of claim 1, wherein:
the material includes a biological material.

10. The composition of claim 9, wherein:
the biological material includes a biological tissue.

11. The composition of claim 9, wherein:
the biological material includes a biological cell.

12. The composition of dam 9, wherein:
the biological material includes a biological organ.

13. The composition of dam 9, wherein:
the biological material includes a biological system.

14. The composition of claim 1, wherein:
the measure of preservative includes alcohol.

15. The composition of claim 1, wherein:
the measure of preservative is configured to sterilize and preserve the amount of potable water existing in the liquid crystalline state.

16. A composition prepared by a process comprising the steps of:
forming an amount of potable water in such a way that the amount of potable water exists in a liquid crystalline state;
resonating the amount of potable water existing in the liquid crystalline state with a material; and
imparting an electromagnetic frequency, produced by an electromagnetic information source, to the amount of potable water while the amount of potable water is converted into the liquid crystalline state in such a way that the structured water was imprinted with and retains the coherent energy pattern that was received from the electromagnetic information; and
mixing a measure of preservative with the amount of potable water existing in the liquid crystalline state.

17. A method of preparing a composition, the method comprising:
resonating an amount of potable water existing in a liquid crystalline state, with a material; and
imparting an eectrornasnetic frequency, produced by an electromagnetic information source, to the amount of potable water while the amount of potable water is converted into the liquid crystalline state in such a way that the structured water was imprinted with and retains the coherent energy pattern that was received from the electromagnetic information; and
mixing a measure of preservative with the amount of potable water existing in the liquid crystalline state.
